(19) United States
(12) Patent Application Publication
(10) Pub. No.: US 2019/0298761 A1
(43) Pub. Date: Oct. 3, 2019

(54) DEVICE FOR PRODUCING AQUEOUS LIQUID HAVING FREE AVAILABLE CHLORINE (FAC)

(71) Applicant: Adept Water Technologies A/S, Skovlunde (DK)

(72) Inventors: Stine Skotte BJERREGAARD, Tikøb (DK); Poul FOGH, Albertslund (DK); Michael Reidtz WICK, Solrød Strand (DK)

(73) Assignee: Adept Water Technologies A/S, Skovlunde (DK)

(21) Appl. No.: 16/468,149

(22) PCT Filed: Dec. 15, 2017
(86) PCT No.: PCT/EP2017/083033

§ 371 Date: Jun. 10, 2019

(30) Foreign Application Priority Data
Dec. 15, 2016 (EP) 16204315.2

(51) Int. Cl. A61K 33/20; A61L 2/00; C25B 15/02; C25B 1/26; C02F 1/467; C02F 1/461; C02F 1/00; C02F 1/42

(52) U.S. Cl. CPC A61K 33/20; A61L 2/0088; C25B 15/02; C25B 1/26; C02F 2103/026; C02F 1/46104; C02F 1/001; C02F 1/42; A61L 2202/11; C02F 1/4674

(57) ABSTRACT

The present disclosure relates to device suitable for producing an aqueous liquid having a desired concentration of FAC wherein the device is adapted to receive municipal water from a municipal water supply, and for the aqueous liquid having the desired concentration of FAC to exit the device from an exit section, wherein the device comprises, a container, a mixing unit, a pumping means, an electrochemical chamber, a flow signal unit, a sterile filter unit, connecting means for transporting liquids, and a control system. In particular, water for disinfection in a desired concentration of FAC can be made using the device.

### FIELD OF THE INVENTION

The present invention relates to a device suitable for producing an aqueous liquid having a desired concentration of free available chlorine. The invention also concerns a method of preparing aqueous liquid having a desired concentration of free available chlorine. Such aqueous liquid having free available chlorine may be used for different purposes, and in particular, may be used for treatment of wound infections.

### BACKGROUND OF THE INVENTION

Hypochlorous acid (HOCl) is well known in the art as an effective biocide and harmless chemical to people and it have been used as a disinfecting agent against various bacteria and viruses. HOCl is shown to be effective in killing many different microorganisms such as Escherichia coli, Pseudomonas aeruginosa, Staphylococcus aureus etc. even low concentration of it is applied. CA2679276 relates to a manufacturing method of sterilized normal saline for medical application, where the low concentration between 0.17 ppm and 6 ppm of HOCl is constantly controlled.

US2013261534 provides a portable/ on-site treatment apparatus for generating and delivering a treatment solution (electrolyzed water) as well as a mechanism for applying the solution to a wound, tissue, bone or surgical cavity for treatment. The apparatus also comprises a mechanism that heats and maintains the temperature of the solution at about body temperature. Further, the electrolysis cell is divided by a membrane, which is a mandatory element of the cell disclosed in US2013261534.

In WO2005094904 and WO2008041031 by Forum Bioscience Holdings Limited, relates to disinfectant solutions/electrolyzed water for killing bacteria and viruses. This invention comprises electrochemically activated water obtainable from electrolysis of NaCl-solutions (brine) with a chlorine (Cl₂) content of no more than 8 ppm.

US2015044144 relates to a disinfectant solution, comprising a super oxidized solution and a colorant, where the colorant is potassium permanganate. The pH of the disinfectant solution is of 3-9 and the concentration of the free chlorine is of 5 ppm-1000 ppm. This solution is stable for 90 days-2 years and has an application in wound care.

US2005196462, US2006235350, EP2330081, US2012251631, US2016045547, US2012207853 and US2010166809 relates to methods of treating a variety of conditions such as skin ulcers, impaired or damaged tissue, peritonitis and sinusitis in patients and biofilm formation, respectively, by administering an oxidative reduction potential (ORP) water solution. The solution is a normal saline containing 0.9% NaCl and has a pH of about 4.5-7.8 and free chlorine species of about 100, but also in concentrations ranging from 10 to 400 ppm.

EP2330081 also provides a process for producing the solution using an electrolytic cell containing anode chamber, cathode chamber, and a salt solution chamber. This device contains a membrane chamber.

CA2829931, US2015196590, US2015231173 and US2015118324 are manufactured by PuriCore Inc., provides a stabilized hypohalous acid solution or/and methods of making the stabilized hypohalous acid solution or/and methods of use for disinfecting mammalian tissue, including wounds among other uses. The free chlorine content of the solution is about 10-10,000 ppm and the pH is 4.0-7.5 wherein the free chlorine and the pH are stable for 6 months. A gel, cream, or foam formulations of the solution is provided in this invention.

US2004060815 relates to a method and apparatus to provide electrochemical solution of 100-250 ppm free chlorine at a pH of 5 and 7. The application has found that a saline solution of a concentration of <1% NaCl and more preferably 0.3% is suitable for the application as biocidal solution.

US2006278585 relates to a device for producing a biocidal saline solution having a pH of 6.2-6.5 and a free chlorine content of 150-420 ppm achieved by a concentration of NaCl of 3.5 grams per liter of softened water.

US2006169575 provides oxidative mixed water with high power of killing microorganisms and healing wound. The water is with a pH around 7.4 ranging from weak acidity to weak alkalinity and is produced by a three-compartment device. Saline with the concentration of 0.05 wt % is fed to both the anode and cathode compartment at the flow rate of 1 liter per minute.

US2012164235 provides a hydrogel formation comprising oxidative reduction potential (ORP) water solution. This invention further provides a method for promoting wound healing using the hydrogel formation. The HOCl concentration is about 50-170 ppm at a pH of about 5.0-8.5.

WO03026679 relates to a saline solution for clinical or cosmetic use. The invention involves replacing some of the sodium ions in normal saline solution with a defined quantity of potassium ions as it has been found to enhance the properties of the solution. Thus, the solution comprises potable or purified water of sodium ions, 0.6-0.9% w/v sodium chloride and 0.001-0.3% potassium ions.

WO2015082937 relates to a composition, formulation, method of manufacture of medical treatment preparations of ultrapure stabilized hypochlorous acid of 80-100 ppm. The hypochlorous acid is produced by adding HCl to tap water or purified water. The pH of the solution is ranging from 3.5-6.3. The flow rate of less than 15 l/h of reactant into the electrolysis chamber is controlled.

US2009148342, US2007134127, US2005232847 and US2008003171 relate to the same manufacture (The Clorox Company P.O.) and provide compositions and methods of producing diluted hypohalous acid and hypohalous acid vapor. The solution of these inventions is used to treat surfaces such as human surfaces, hospital surfaces, food contact surfaces etc. The solution of US2009148342 comprises purified water 0.3 g/l salt, and a free chlorine concentration of 40-400 ppm. The pH of the solution is adjusted by an adjusting agent.

US2003185704 describes physiologically-balanced, acidic solution and methods for use of the solutions, including a specialized bandage. The author described that the solution is nontoxic and has antibacterial properties. The solution is prepared by the electrolysis of a solution comprising a mixture of an inorganic salt such as fluoride, chloride, bromide, or iodide in an amount of 0.4-20.4 g/L. The solution has a pH of 2-5 and hypohalous acid concentration is about 10-200 ppm.

EP1728521 is an invention wherein a concentration adjusting unit is equipped with a flow rate adjusting valve for adjusting the flow rate of humidifying water in accordance with the conductivity of tap water. This invention uses another type of apparatus without a membrane. In CA2721310, US2010285151, US2015231173, and US2014328945 the conductivity is discussed, but these inventions neither providing methods to produce HOCl by mixing chemicals or utilizing another type of apparatus. In general, the solutions of these inventions are acidic at a pH of about 2-4 and the concentrations of hypochlorous acid solution are high of 7000 ppm and 1000 ppm.

In US2012164235, the oxidative reductive potential water is heated to 50° C., but the deference is that this water is utilized to produce a hydrogel formulation. This invention focused on the method not on the apparatus for producing the formulation.

CA2383923 is an invention relates to a method for producing a sterile medical solution at a pH of 2.6-5 by mixing at least two components which are heat sterilized in the heat sterilizing device.

US2014328945 provides a method for stabilizing electrochemically generated solution with disinfecting properties having a stabilizing amount of Dissolved Ionic Compounds (DIC), a HOCl concentration of 10 -1000 ppm, and a pH from about 4.0 to about 7.5. This invention also relates to a method to stabilize hydrogel formulations.

US2008264778 and WO2009011841 are manufactured by Ceramatec, INC. Both inventions provide on-site membrane electrolytic cells to generate and dispense a cleansing agent. Both the inventions are provided by the concentration of sodium chloride in the aqueous solution of this invention is below its saturation limit in water preferably between 1%- 26% w/v of the solution.

### SUMMARY OF THE INVENTION

The present inventors have designed a device which overcome many of the daily problems experienced when dealing with production of aqueous liquid having a desired concentration of free chlorine. In particular, the device of the present invention may be used to produce normal saline on site at low concentration of free chlorine for use extra venously with low risk of infection. Furthermore, a higher concentration up to 200 ppm of free chlorine can also be provided by the present invention to kill microorganisms, e.g. bacteria. Even higher concentrations up to 1000 ppm of free chlorine can also be provided by the present invention, for instance by regulating the current as the skilled person knows that there is a relationship between current and chlorine concentration. Further advantages of the device of the present invention is that it can be a stand-alone device and is portable and can be used in for instance different places in a hospital facility. In preferred embodiments, the electrolysis chamber of the portable device having no membrane for producing a disinfecting solution (sterile Normal Saline Solution with HOCl of 0.3-200 ppm at a pH of 5.5-6.5) is particularly suitable for adjuvant wound therapy, wherein the conductivity is constant, the current and charge is measured and regulated, and the solution is heated to a temperature of about 32 to 35° C.

The present invention relates to a device (10, 100) suitable for producing an aqueous liquid having a desired concentration of free available chlorine (FAC) wherein the device is adapted to receive municipal water from a municipal water supply (12, 112), and for the aqueous liquid having the desired concentration of FAC to exit the device from an exit section (14, 114), comprising
a) a container (16) adapted to contain saline water of a desired salt concentration,
b) a mixing unit (18) adapted for mixing the saline water from the container with the municipal water,
c) a pumping means (20) adapted to move the saline water from the container to the mixing unit,
d) or as an alternative to a)-c) an isotonic water generator (102),
e) an electrochemical chamber (22, 122) comprising a pair of electrodes for providing electrolysis of the aqueous liquid entering the chamber from the mixing unit, wherein the pair of electrodes are adapted to receive current from a current supply (24, 124),
f) a flow signal unit (26, 126, 28) for measuring a volume velocity of the aqueous liquid entering the electrochemical device and/or the mixing unit, and capable of providing volume velocity data, or if the velocity of the aqueous liquid and salt concentration hereof are fixed and known, then the flow signal unit (126) may be omitted,
g) a sterile filter unit (30, 130) adapted to receive the aqueous liquid from the electrochemical chamber, and for the filtered aqueous liquid to exit the sterile filter unit,
h) (i) a connecting means for transporting (32) municipal water from the supply to the mixing unit, for transporting (34) saline water to the mixing unit, for transporting (36) mixed water to the electrochemical chamber, for transporting (38) the aqueous liquid from the electrochemical chamber to the sterile filter unit, and for transporting (40) the aqueous liquid from the sterile filter to the exit section (14, 114), or alternatively (ii) in d) a connecting means for transporting (132) isotonic water to the electrochemical chamber (122), for transporting (138) the aqueous liquid from the electrochemical chamber to the sterile filter unit (130), and for transporting (140) the aqueous liquid from the sterile filter (130) to the exit section (114) and
j) a control system (42, 142) adapted to communicate with one or more selected from the group consisting of the container, the mixing unit, the pumping means, the electrochemical chamber, the flow signal unit, the current supply, and the exit section.

In another aspect the present invention relates to a device (100) suitable for producing an aqueous liquid having a desired concentration of free available chlorine (FAC) wherein the device is adapted to receive municipal water from a municipal water supply (112), and for the aqueous liquid having the desired concentration of FAC to exit the device from an exit section (114), comprising
i) an isotonic water generator (102),
ii) an electrochemical chamber (122) comprising a pair of electrodes for providing electrolysis of the aqueous liquid entering the chamber from the mixing unit, wherein the pair of electrodes are adapted to receive current from a current supply (124),
iii) a flow signal unit (126) for measuring a volume velocity of the aqueous liquid entering the electrochemical device and/or the mixing unit, and capable of providing volume velocity data, or if the velocity of the aqueous liquid and salt concentration hereof are fixed and known, then the flow signal unit (126) may be omitted,
iv) a sterile filter unit (130) adapted to receive the aqueous liquid from the electrochemical chamber, and for the filtered aqueous liquid to exit the sterile filter unit,
v) a connecting means for transporting (132) isotonic water to the electrochemical chamber (122), for transporting (138) the aqueous liquid from the electrochemical chamber to the sterile filter unit, and for transporting (140) the aqueous liquid from the sterile filter to the exit section (114), and
vi) a control system (142) adapted to communicate with one or more selected from the group consisting of the electrochemical chamber, the flow signal unit, the current supply, and the exit section.

In an embodiment the step iii) concerns the flow signal unit (126) for measuring a volume velocity of the aqueous liquid entering the electrochemical device and/or the mixing unit, and capable of providing volume velocity data. In an alternative of the step iii) the velocity of the aqueous liquid and salt concentration hereof are fixed and known, and there is no flow signal unit (126).

The present invention also concerns a device suitable for producing an aqueous liquid having a desired concentration of free available chlorine (FAC) wherein the device is adapted to receive municipal water from a municipal water supply, and for the aqueous liquid having the desired concentration of FAC to exit the device from an exit section, comprising
a) a container adapted to contain saline water of a desired salt concentration,
b) a mixing unit adapted for mixing the saline water from the container with the municipal water,
c) a pumping means adapted to move the saline water from the container to the mixing unit,
d) an electrochemical chamber comprising a pair of electrodes for providing electrolysis of the aqueous liquid entering the chamber from the mixing unit, wherein the pair of electrodes are adapted to receive current from a current supply,
e) a flow signal unit for measuring a volume velocity of the aqueous liquid entering the electrochemical device and/or the mixing unit, and capable of providing volume velocity data,
f) a sterile filter unit adapted to receive the aqueous liquid from the electrochemical chamber, and for the filtered aqueous liquid to exit the sterile filter unit,
g) a connecting means, such as tubes, pipes or the like made of plastic, metal or the like, for transporting municipal water from the supply to the mixing unit, for transporting saline water to the mixing unit, for transporting mixed water (aqueous liquid) to the electrochemical chamber, for transporting the aqueous liquid from the electrochemical chamber to the sterile filter unit, and for transporting the aqueous liquid from the sterile filter to the exit unit, and
h) a control system adapted to communicate with one or more selected from the group consisting of the container, the mixing unit, the pumping means, the electrochemical chamber, the flow signal unit, the current supply, and the exit section.

In one embodiment of the present invention the device further comprises a pre-filter unit adapted to receive the municipal water, and connecting means for transporting the filtered municipal water to the mixing unit.

In a further embodiment of the present invention the device further comprises a water softener unit, wherein the water softener unit is adapted to receive filtered municipal water, and connecting means for transporting the softened and filtered municipal water to the mixing unit. Typically, the water softener unit is selected from an ion exchange unit, such as an ion exchange unit adapted to lower pH. In a further embodiment, the water softener unit is integrated in the pre-filter unit.

In a still further embodiment of the present invention the device further comprises a activated carbon filter unit, optionally integrated in the pre-filter unit, wherein the activated carbon filter unit is adapted to receive filtered municipal water, and optionally softened water, and connecting means for transporting the activated carbon filtered municipal water to the mixing unit.

In a further embodiment of the present invention the device further comprises a dosing section at the exit section or a dosing section remote from the exit section and connecting means for transporting the aqueous liquid from the exit section to the dosing section.

In a still further embodiment of the present invention the device further comprises a user interface wherein the control system is adapted to communicate with the user interface. A typical, user interface is an integrated part of the device of the present invention, but may also be remote and selected from a computer, an ipad, a smart phone, a notebook, and a mac).

In a further embodiment of the present invention the device is suitable for producing an aqueous liquid for disinfection containing a desired concentration of FAC. A preferred concentration of FAC is from 0.3 ppm to 1000 ppm FAC, such as from 0.3 ppm to 200 ppm FAC, for instance from 0.3 ppm to 120 ppm FAC.

In a still further embodiment the device of the present invention is a stand-alone device.

In a further embodiment of the present invention the device is adapted to operate at a volume velocity from 0.5 to 10 L aqueous liquid per minute, typically from 0.5 to 2 L/minute, such as about 1 L/minute.

In a still further embodiment the device of the present invention further comprises a backflow prevention device, such as conforming to EN 1717, adapted to receive municipal water from the supply and connecting means for transporting the municipal water to the mixing unit, optionally via the pre-filter unit, the water softener unit and/or the activated carbon unit. Typically, such backflow prevention device is conforming to EN 1717.

In a further embodiment of the present invention a flush system is incorporated in the dosing section. In particular, this flush system will allow the aqueous liquid to be drained, when for instance, the temperature has dropped below a threshold temperature, or after a predetermined interval, or if or a new FAC range has been selected. A typical threshold temperature is 32° C.

In a still further embodiment of the present invention the FAC in the aqueous liquid has a concentration suitable for use in a method of adjuvant wound therapy.

In a further embodiment of the present invention the flow signal unit is located between the mixing unit and the electrochemical device.

In a still further embodiment of the present invention a further flow signal unit is located between the container and the mixing unit.

In a further embodiment of the present invention a further non-return valve means is located in the connection means between the container and the mixing unit. Non-return valves are inserted in the brine supply string and before the electrolysis section, allowing wetted materials before the electrolysis string to be made of non-chlorine resistant materials.

In a still further embodiment of the present invention the electrochemical chamber is without any membrane.

In a further aspect, the present invention relates to use of the device of the present invention for preparing an aqueous liquid having a desired concentration of FAC for treatment of infection in wounds. Typically, the concentration of FAC is from 0.3 to 1000 ppm.

Preferably the concentration of FAC is from 0.3 to 200 ppm. A further range is the concentration of FAC from 0.3 to 120 ppm.

In a further aspect, the present invention relates to a method of preparing an aqueous liquid having a desired concentration of free available chlorine (FAC) comprising the steps of,
a) supplying municipal water to the device of the present invention, wherein the device contains saline water in the container,
b) supplying current to the device,
c) mixing municipal water and saline water in the mixing unit, and leading the aqueous liquid to the electrochemical chamber,
d) adjusting water and liquid flows and dosing to provide the aqueous liquid having the desired concentration of FAC, and
e) collecting the aqueous liquid having the desired concentration of FAC at the exit section.

In an embodiment of the method of the present invention the current is adjusted proportionally to the liquid flows. In a further embodiment, the current is delivered in pulses.

In a further embodiment of the method of the present invention the concentration of FAC at the exit section is selected from 0.3 ppm to 1000 ppm, such as from 0.3 ppm to 200 ppm, such as from 0.3 ppm to 120 ppm, e.g. from 10 ppm to 100 ppm.

In a further embodiment of the method of the present invention the pH of the water and the aqueous liquid in the device is between 5 and 8.5, such as between 5.5 and 7.5.

Preferably, the pH of the water and the aqueous liquid in the device is between 5.5 and 7, such as between 5.5 and 6.5.

In a still further embodiment of the method of the present invention the volume velocity of the device is adjusted from 0.5 to 10 L aqueous liquid per minute. In a further embodiment of the method of the present invention the volume velocity of the device is adjusted from 0.5 to 2 L aqueous liquid per minute, typically 1 L/minute.

In a further embodiment of the method of the present invention the salt concentration of the saline water is from 10% w/w to saturated, such as 15% w/w to saturated.

In a still further embodiment of the method of the present invention the current is delivered in pulses, where the peak current is 10 A and the mean current, for the volume velocity of 0.5 to 2 L aqueous liquid per minute, is 5-7 A and the voltage is 6-10V, such as 8V, to deliver the aqueous liquid having the desired concentration of 100 ppm FAC.

In a further embodiment of the method of the present invention the municipal water from the supply is drinking water according to latest WHO guidelines for drinking water quality.

In a still further embodiment of the method of the present invention the municipal water and/or saline water from the container and/or aqueous liquid is/are heated above room temperature during the preparation, such as by mixing water and saline water by different temperatures or by using a heating system for heating the aqueous liquid before entering the electrochemical chamber.

In a further embodiment of the method of the present invention the temperature of the aqueous liquid entering the electrochemical chamber is in the range of 30° C.-35° C.

In a still further embodiment of the method of the present invention the municipal water entering the mixing unit has been through the pre-filter and the water softener unit.

In a further embodiment of the method of the present invention the water softener unit additionally is used to lower the pH of the municipal water.

In a still further embodiment of the method of the present invention the additional flow signal unit located between the container and the mixing unit is used to partly control the dosing speed of the saline water from the container and the electrical current used for the electrolysis, and wherein automated setting or user input is used to partly control the dosing speed of the saline water from the container and the electrical current used for the electrolysis.

In a further embodiment of the method of the present invention the voltage and/or current is measured in the electrochemical chamber, using the constant characteristics of the chamber to calculate the conductivity of the aqueous liquid passing through the chamber. In a typical embodiment, a constant conductivity of the aqueous liquid is maintained.

In a still further embodiment the method of the present invention further comprises a dosing section separate from the exit section or a dosing section remote from the exit section and connecting means for transporting the aqueous liquid from the exit section to the dosing section.

In a further embodiment of the method of the present invention a flush system is incorporated in the dosing section, the flush system automatically leading a predetermined amount of aqueous liquid to drain before allowing a user to draw the aqueous liquid. Typically, the predetermined amount is from 100-500 mL of aqueous liquid. The user is typically a physician or nurse. The volume is proportional to how much should be exchanged at stagnation (when heating) and is dependent on where the heater is located.

In a still further embodiment of the method of the present invention a flush system is incorporated in the dosing section, the flush system automatically flushing for a preset period of time, before allowing a user, such as a physician or nurse, to draw the aqueous liquid.

In a further embodiment of the method of the present invention the container and pumping means in operation produce a selected range of salt concentrations in the aqueous liquid leaving the mixing unit from 0.01% w/w to 0.95% w/w at a water flow of 1 L/minute.

In a still further embodiment of the method of the present invention the current applied to the electrolysis and timing generates from 40-200 ppm of chlorine in a normal saline liquid (0.9% NaCl), wherein the concentration (ppm) of chlorine is selected and kept constant ±10%.

In another embodiment of the method of the present invention the current applied to the electrolysis and timing generates up to 1 ppm of chlorine in a normal saline liquid, wherein the concentration (ppm) of chlorine is selected and kept constant ±75%.

In a further embodiment of the method of the present invention the current applied to the electrolysis and timing generates from 5-40 ppm of chlorine in an aqueous liquid containing 0.01-0.25% salt (NaCl), wherein the concentration (ppm) of chlorine is selected and kept constant ±75%.

In a still further embodiment of the method of the present invention the municipal water supplied is drinking water which is mixed with saline water from the container, wherein the saline water comprises high-purity sodium chloride dissolved in mineral-free water.

In another embodiment of the method of the present invention the municipal water supplied is drinking water which is mixed with saline water from the container, wherein the saline water comprises various salts and the device is adjusted to an osmotic pressure of approximately 308 mOsmol/L, using a conversion from conductivity to osmotic pressure, where the error from non-sodium and non-chloride salts present constitute less than 1%.

In a further embodiment of the method of the present invention the aqueous liquid produced is for use in adjuvant wound therapy.

In a still further embodiment of the method of the present invention the current is regulated to a constant level using a constant current generator circuit, and the voltage varies with the area of the pair of electrodes. Typically, the voltage is form 3.7-5.7 V, and will depend on the surface area of the electrode plates and the distance between the plates.

In a further embodiment of the method of the present invention the pumping means for transporting the salt water from the container to the mixing unit is operated in time intervals.

In a still further embodiment of the method of the present invention a non-return valve means is inserted in the connecting means for transporting saline water to the mixing unit and/or in the connecting means for transporting aqueous liquid to the electrochemical chamber.

Further objects and advantages of the present invention will appear from the following description, and claims.

### BRIEF DESCRIPTION OF DRAWINGS

FIG. 1 illustrates an embodiment of the device of the present invention, which may be a portable stand-alone device and incorporate all of the units and section illustrated.

FIG. 2 illustrates another embodiment of the device of the present invention, which may be a portable stand-alone device and incorporate all of the units and section illustrated.

### DESCRIPTION OF THE INVENTION

There are many advantages of the present invention in a broad context as well as further even more advantages aspects of the embodiments. The device of the present invention may be used to produce normal saline on site at low concentration of free chlorine for use extra venously with low risk of infection. Furthermore, a higher concentration up to 200 ppm of free chlorine can also be provided by the present invention to kill microorganisms, e.g. bacteria. Even higher concentration up to 1000 ppm of free chlorine can be provided by the present invention.

Further additional advantage of the present invention is the high capacity, in which the water flow/ aqueous liquid flow in the device of the present invention is for instance, 1 liter/minute. Maintaining a constant conductivity of the aqueous liquid will secure high quality of the liquid. The high capacity of the device of the present invention of 1 liter/minutes and the ease of use are additional advantages that will enhance wound washes, which then promote wound healing.

Adjusting the preferred temperature of the aqueous liquid in a range between 30° C.-35° C. is possible during the method of preparation using the present device and can for instance, be obtained by mixing of hot and cold water or by a heating element. In a further embodiment, a heating element is applied to the device. Optionally such heating element is integrated in the device, heating the aqueous liquid flowing in the connection between the mixing unit and the electrochemical chamber. Delivering the liquid at a temperature in the range of 30° C.-35° C. is an additional advantage of the device of the present invention especially when the liquid is used for wound healing.

Furthermore, when a water softener is used it may be a softener additionally reducing and keeping the preferred pH between 5.5 and 7. This ensures a high fraction of HOCl and thus enhanced biocidal effect.

Furthermore, the device of the present invention and in particular the electrolysis chamber (cell) provides a simpler solution compared to known devices and methods of preparing aqueous liquid containing FAC, and it can be operated when the electrolysis cell is not divided by a membrane.

The present invention offers the preparation of a neutral liquid solution and the conventional electrochemical activation of processes may draw a current of up to 10 A, which may be regulated or pulsed.

Many prior art aqueous liquids prepared according to known methods have stability problems. The present invention overcomes this stability problem because the aqueous liquid containing FAC is produced by the portable device of the present invention, and can immediately be applied for medical treatment on site, for instance, at hospitals or emergency rooms. Furthermore, it is an additional advantage that the device of the present invention can heat the liquid especially when applying it in wound care. Moreover, the direct use of the aqueous liquid leads to avoiding short lifetime of the oxidizing species therein.

Some prior art methods disclose mixing of water from the anode and cathode in different volumes, which adds a further step to the process, and this step is avoided using the device of the present invention, since the liquids are already mixed in the electrolytic cell. Mixing the anode water and the cathode water according to prior art methods is performed in 30 minutes or 300 minutes, and this time can be saved using the device of the present invention, since both the cathode and the anode water is produced and mixed in the electrolytic cell and can immediately be applied on site, as explained above.

The aqueous liquid containing FAC as produced by the device used in the method of the present invention can be applied for the same purpose as in prior art without need for adding other chemicals, such as bicarbonate or carbonate.

The device of the present invention can produce a saline solution from saturated salt solution and facilitates the regulation of chloride input to the cell, thereby controlling the salinity.

There is no need for a conductivity probe by utilizing the device of the present invention, since the conductivity of the aqueous liquid can be measured in the electrolysis cell and can be maintained constant.

Many prior art methods are conducted with use of different parameters which must be adjusted during use, such as the back pressure on the cell, whereas the device of the present invention avoids such adjusting during the use of the device by measuring the flow of liquid/water and adjusting the dosing of brine and electrical energy.

As disclosed it is possible to select and produce the concentration of free chlorine in the interval of 0.3-1000 ppm, with the method of the present invention, and it is therefore a particularly advantage to have a sterile filter to resolve the microbiological issues that may arise, when a low concentration of free available chlorine is desired.

The applications of the present invention are widely and include preparing aqueous liquid with FAC for adjuvant wound therapy.

A further advantage of the present invention is to avoid addition of chemicals since the device and method only requires use of municipal water, e.g. tap water, and sodium chloride in a concentration of 0.01%-0.95% w/w.

The aqueous liquid produced by the method of the present invention is made by a simpler technique and have an additional advantage of offering a sterilized aqueous liquid at a temperature equivalent to the temperature of human body and therefore is suitable for medical applications.

A further advantage of the present invention is the controlling of speed of salt/saline water pumping means and the electrical current.

Another advantage of the present invention is the possible adjustment of pH inside the electrochemical chamber/cell without addition of chemicals, and further where the aqueous liquid has a FAC concentration suitable for wide disinfecting applications, including medical treatment, such as adjuvant wound therapy.

The device of the present invention when in operation and connected to municipal water offers brine dosing control based on a measurement of the conductivity, which is calculated by measuring the current in the cell using the constant characteristics of the cell. Controlling the brine dosing and maintaining a constant conductivity of the aqueous liquid passing through the cell is a further property offered by the present invention.

The present invention concerns a device suitable for producing an aqueous liquid having a desired concentration of free available chlorine (FAC) wherein the device is adapted to receive municipal water from a municipal water supply, and for the aqueous liquid having the desired concentration of FAC to exit the device from an exit section, comprising
a) a container adapted to contain saline water of a desired salt concentration,
b) a mixing unit adapted for mixing the saline water from the container with the municipal water,
c) a pumping means adapted to move the saline water from the container to the mixing unit,
d) an electrochemical chamber comprising a pair of electrodes for providing electrolysis of the aqueous liquid entering the chamber from the mixing unit, wherein the pair of electrodes are adapted to receive current from a current supply,
e) a flow signal unit for measuring a volume velocity of the aqueous liquid entering the electrochemical device and/or the mixing unit, and capable of providing volume velocity data,
f) a sterile filter unit adapted to receive the aqueous liquid from the electrochemical chamber, and for the filtered aqueous liquid to exit the sterile filter unit,
g) a connecting means, such as tubes, pipes or the like made of plastic, metal or the like, for transporting municipal water from the supply to the mixing unit, for transporting saline water to the mixing unit, for transporting mixed water (aqueous liquid) to the electrochemical chamber, for transporting the aqueous liquid from the electrochemical chamber to the sterile filter unit, and for transporting the aqueous liquid from the sterile filter to the exit unit, and
h) a control system adapted to communicate with one or more selected from the group consisting of the container, the mixing unit, the pumping means, the electrochemical chamber, the flow signal unit, the current supply, and the exit section.

With the device of the present invention the desired concentration of FAC can be determined and set at any desired concentration, once the device is in operation and connected to the municipal water supply and saline water is in the container. Also, since the device is portable and works as a stand-alone unit it can be used in many places and in different situations, and offers multiple solutions as described herein.

In one embodiment of the present invention the device further comprises a pre-filter unit adapted to receive the municipal water, and connecting means for transporting the filtered municipal water to the mixing unit. Preferred pre-filters are selected from the group consisting of any suitable mechanical filter.

In a further embodiment of the present invention the device further comprises a water softener unit, wherein the water softener unit is adapted to receive filtered municipal water, and connecting means for transporting the softened and filtered municipal water to the mixing unit. Typically, the water softener unit is selected from an ion exchange unit, such as an ion exchange unit adapted to lower pH. Preferred water softeners are selected from the group consisting of a cartridge type generated with hydrochloric acid, the cartridge type generated with sodium chloride, a regenerative type with hydrochloric acid as regenerative agent, and a regenerative type with sodium chloride as regenerative agent.

In a further embodiment, the water softener unit is integrated in the pre-filter unit.

In a still further embodiment of the present invention the device further comprises an activated carbon filter unit, wherein the activated carbon filter unit is adapted to receive filtered municipal water, and connecting means for transporting the activated carbon filtered municipal water to the mixing unit. In a further embodiment, the activated carbon filter unit is integrated in the pre-filter unit. In a still further embodiment the activated carbon filter unit is adapted to receive filtered municipal water and softened water. A preferred activated carbon filters is an activated carbon filter integrated in a water softener. Alternatively, a separate filter is located before or after the water softener.

In a further embodiment of the present invention the device further comprises a dosing section at the exit section and connecting means for transporting the aqueous liquid from the exit section to the dosing section. In another embodiment of the present invention the device further comprises a dosing section remote from the exit section and connecting means for transporting the aqueous liquid from the exit section to the dosing section. Typical dosing sections are selected from a T-element having a non-return valve, an injector, optionally followed by a tube section with baffles to ensure suitable mixing.

In a still further embodiment of the present invention the device further comprises a user interface wherein the control system is adapted to communicate with the user interface. A typical, user interface is an integrated part of the device of the present invention, but may also be remote and selected from a computer, an ipad, a smart phone, a notebook, and a mac.

In a further embodiment of the present invention the device is suitable for producing an aqueous liquid for disinfection containing a desired concentration of FAC. A preferred concentration of FAC is from 10 ppm to 200 ppm FAC. Another preferred concentration of FAC is from 10 ppm to 120 ppm FAC.

In a still further embodiment the device of the present invention is a stand-alone device. In a further embodiment, the device of the present invention is a portable and stand-alone device.

In a further embodiment of the present invention the device is adapted to operate at a volume velocity from 0.5 to 10 L aqueous liquid per minute. In a still further embodiment of the present invention the device is adapted to operate at a volume velocity from 0.5 to 2 L aqueous liquid per minute, typically 1 L/minute.

In a still further embodiment the device of the present invention further comprises a backflow prevention device adapted to receive municipal water from the supply and connecting means for transporting the municipal water to the mixing unit, optionally via the pre-filter unit, the water softener unit and/or the activated carbon unit. In one embodiment, the pre-filter is present. In another embodiment, the water softener unit is present. In a further embodiment, the activated carbon unit is present. Also, combinations of the pre-filter unit, the water softener unit and the activated carbon unit are contemplated, such as a combination of the pre-filter and the water softener, or the pre-filter and the activated carbon unit, or all three units. Typically, such backflow prevention device is conforming to the standard EN 1717.

In a further embodiment of the present invention a flush system is incorporated in the dosing section.

In a still further embodiment of the present invention the FAC in the aqueous liquid has a concentration suitable for use in a method of adjuvant wound therapy.

In a further embodiment of the present invention the flow signal unit is located between the mixing unit and the electrochemical device. Such flow signal unit is typically selected from an impeller type, a mass flow meter, a magnetic type, a gear type sensor, a coriolis type sensor.

In a still further embodiment of the present invention a further flow signal unit is located between the container and the mixing unit.

In a further embodiment of the present invention a non-return valve means is located between the container and the electrochemical chamber. In another embodiment of the present invention a non-return valve means is located between the container and the mixing unit.

In a still further embodiment of the present invention the electrochemical chamber is without any membrane. Typically, such electrochemical chamber is containing a pair of parallel and symmetrically arranged perforated electrode plates, which electrodes are also suitable for use in the present invention. Thus, in a further embodiment the electrode comprises a pair of parallel and symmetrically arranged perforated electrode plates having a suitable distance, wherein each pair is optionally fitted with a fuse, wherein a suitable current density is applied, and wherein the plates are made of a conductive material and are arranged in a perpendicular plane. Typically, the pair of parallel and symmetrically arranged perforated electrode plates has/have a distance selected from 1-5 mm, such as 1, 2, 3, 4 or 5 mm and combinations thereof. The electrodes are arranged in pairs that may have the same distance between the plates or may have different distance between the plates, if more than one pair of electrodes is present. Typically, from 1 to 11 pairs of parallel and symmetrically arranged perforated electrode plates are present, such as 1-10, 2-9, 3-8, 4-7, or 5-6 pairs of parallel and symmetrically arranged perforated electrode plates.

In a further aspect, the present invention relates to a method of preparing an aqueous liquid having a desired concentration of free available chlorine (FAC) comprising the steps of,
a) supplying municipal water to the device of the present invention, wherein the device contains saline water in the container,
b) supplying current to the device,
c) mixing municipal water and saline water in the mixing unit, and leading the aqueous liquid to the electrochemical chamber,
d) adjusting water and liquid flows and dosing to provide the aqueous liquid having the desired concentration of FAC, and
e) collecting the aqueous liquid having the desired concentration of FAC at the exit section.

In a still further aspect, the present invention relates to a method of preparing an aqueous liquid having a desired concentration of free available chlorine (FAC) comprising the steps of,
a) supplying municipal water to the device of any one of claims 1-20, wherein the device contains saline water in the container, or the isotonic water generator is present,
b) supplying current to the device,
c) mixing municipal water and saline water in the mixing unit, and leading the aqueous liquid to the electrochemical chamber, or supplying municipal water to the isotonic water generator,
d) adjusting water and liquid flows, and dosing to provide the aqueous liquid having the desired concentration of FAC, and
e) collecting the aqueous liquid having the desired concentration of FAC at the exit section.

In an embodiment of the method of the present invention the concentration of FAC at the exit section is selected from 0.3 ppm to 1000 ppm. In another embodiment of the method of the present invention the concentration of FAC at the exit section is selected from 0.3 ppm to 200 ppm. In a further embodiment, the concentration of FAC at the exit section is selected from 10 ppm to 100 ppm. In a further embodiment, the concentration of FAC at the exit section is selected from 10 ppm to 120 ppm. In a still further embodiment the concentration of FAC at the exit section is selected from 80 ppm to 200 ppm, such as 80 ppm to 120 ppm. In a further embodiment, the concentration of FAC at the exit section is selected from 0.3 ppm to 2 ppm, such as 0.3 ppm to 1 ppm.

In a further embodiment of the method of the present invention the pH of the water and the aqueous liquid in the device is between 5 and 8. In a further embodiment the pH is between 5.5 and 7.5. Preferably, the pH of the water and the aqueous liquid in the device is between 5.5 and 6.5.

In a still further embodiment of the method of the present invention the volume velocity of the device is adjusted to from 0.5 to 10 liter (L) aqueous liquid per minute. In a further embodiment of the method of the present invention the volume velocity of the device is adjusted to from 0.5 to 2 L aqueous liquid per minute. Typically, 0.75 to 1.5 L aqueous liquid per minute. In one embodiment of the invention the volume velocity of the device is adjusted to about 1 L aqueous liquid per minute.

The salt concentration of the saline water in the container may be selected from any desired concentration such as from 3% w/w to saturated. Typically, the saline water is from 5% w/w to saturated. In a further embodiment of the method of the present invention the salt concentration of the saline water is from 10% w/w to saturated, such as 15% w/w to saturated.

As stated according to the present invention it is possible to prepare any desired concentration of FAC at the exit, which means that by adjusting parameters of the process when using the device, a specified concentration of FAC may be reached. For instance, it may be desired to deliver the aqueous liquid having the desired concentration of 100 ppm FAC. Thus, in one embodiment of the method of the present invention the current is delivered in pulses, where the peak current is 10 A and the mean current, for the volume velocity of from 0.5 to 2 L aqueous liquid per minute, is 5-7 A and the voltage is between 6-10V, to deliver the aqueous liquid having the desired concentration of 100 ppm FAC. Preferably, the current is delivered in pulses, where the peak current is 10 A and the mean current, for the volume velocity of 1 L aqueous liquid per minute, is 5-7 A and the voltage is 8V, to deliver the aqueous liquid having the desired concentration of 100 ppm FAC.

In a further embodiment of the method of the present invention the municipal water from the supply is drinking water according to latest WHO guidelines for drinking water quality.

In a still further embodiment of the method of the present invention the municipal water and/or saline water from the container and/or aqueous liquid is/are heated above room temperature during the preparation, such as by mixing water and saline water by different temperatures or by using a heating system for heating the aqueous liquid before entering the electrochemical chamber.

In a further embodiment of the method of the present invention the municipal water and/or saline water from the container and/or aqueous liquid is/are heated above room temperature during the preparation, by mixing water and saline water by different temperatures before entering the electrochemical chamber.

In a further embodiment of the method of the present invention the municipal water and/or saline water from the container and/or aqueous liquid is/are heated above room temperature during the preparation, by using a heating system for heating the aqueous liquid before entering the electrochemical chamber.

In a further embodiment of the method of the present invention the temperature of the aqueous liquid entering the electrochemical chamber is in the range of 30° C.-35° C. In a still further embodiment of the method of the present invention the temperature of the aqueous liquid leaving the electrochemical chamber is in the range of 30° C.-35° C.

In a still further embodiment of the method of the present invention the municipal water entering the mixing unit has been through the pre-filter and the water softener unit.

In a further embodiment of the method of the present invention the water softener unit additionally is used to lower the pH of the municipal water. Typically, the water softener unit is selected from an ion exchange unit, such as an ion exchange unit adapted to lower pH. Preferred water softeners are selected from the group consisting of a cartridge type generated with hydrochloric acid, the cartridge type generated with sodium chloride, a regenerative type with hydrochloric acid as regenerative agent, and a regenerative type with sodium chloride as regenerative agent.

In a still further embodiment of the method of the present invention the additional flow signal unit located between the container and the mixing unit is used to partly control the dosing speed of the saline water from the container and the electrical current used for the electrolysis, and wherein automated setting or user input is used to partly control the dosing speed of the saline water from the container and the electrical current used for the electrolysis.

In a further embodiment of the method of the present invention the voltage and/or current is measured in the electrochemical chamber, using the constant characteristics of the chamber to calculate the conductivity of the aqueous liquid passing through the chamber. In a typical embodiment, a constant conductivity of the aqueous liquid is maintained.

In a still further embodiment the method of the present invention further comprises a dosing section separate from the exit section and connecting means for transporting the aqueous liquid from the exit section to the dosing section. In a further embodiment, the method of the present invention further comprises a dosing section as part of the exit section. In a further embodiment, the method of the present invention further comprises a dosing section remote from the exit section and connecting means for transporting the aqueous liquid from the exit section to the dosing section. Examples of such remote dosing section may be at the place of treatment where the generating device is placed at a central position, or the generating device is placed on a shelf of a sliding table and the dosing section is on the table.

In a further embodiment of the method of the present invention a flush system is incorporated in the dosing section, the flush system automatically leading a predetermined amount of aqueous liquid to drain before allowing a user to draw the aqueous liquid. Typically, the predetermined amount is from 100-500 mL of aqueous liquid. The user is typically a physician or nurse.

In a still further embodiment of the method of the present invention a flush system is incorporated in the dosing section, the flush system automatically flushing for a preset period of time, before allowing a user, such as a physician or nurse, to draw the aqueous liquid.

In a further embodiment of the method of the present invention the container and pumping means in operation produce a selected range of salt concentrations in the aqueous liquid leaving the mixing unit from 0.01% w/w to 0.95% w/w at a water flow of 1 L/minute.

In a still further embodiment of the method of the present invention the current applied to the electrolysis and timing generates from 40-200 ppm of chlorine in a normal saline liquid (0.9% NaCl), wherein the concentration (ppm) of chlorine is selected and kept constant ±10%.

In another embodiment of the method of the present invention the current applied to the electrolysis and timing generates up to 1 ppm of chlorine in a normal saline liquid, wherein the concentration (ppm) of chlorine is selected and kept constant ±75%.

In a further embodiment of the method of the present invention the current applied to the electrolysis and timing generates from 5-40 ppm of chlorine in an aqueous liquid containing 0.01-0.25% salt (NaCl), wherein the concentration (ppm) of chlorine is selected and kept constant ±75%.

In a still further embodiment of the method of the present invention the municipal water supplied is drinking water which is mixed with saline water from the container, wherein the saline water comprises high-purity sodium chloride dissolved in mineral-free water. Preferably, the purity conforms to the standard EN 14805.

In another embodiment of the method of the present invention the municipal water supplied is drinking water which is mixed with saline water from the container, wherein the saline water comprises various salts and the device is adjusted to an osmotic pressure of approximately 308 mOsmol/L, using a conversion from conductivity to osmotic pressure, where the error from non-sodium and non-chloride salts present constitute less than 1%.

In a further embodiment of the method of the present invention the aqueous liquid produced is for use in adjuvant wound therapy.

In a still further embodiment of the method of the present invention the current is regulated to a constant level using a constant current generator circuit, and the voltage varies with the area of the pair of electrodes. Typically, the voltage is form 3.7-5.7 V.

In a further embodiment of the method of the present invention the pumping means for transporting the saline water from the container to the mixing unit is operated in time intervals. In further embodiments, such time intervals are selected from 1 second of every 3 seconds, or 2 seconds of every 4 seconds, or 1.35 seconds of every 3 seconds, or 5 seconds of every 6 seconds.

In a still further embodiment of the method of the present invention a non-return valve means is inserted in the connecting means for transporting saline water to the mixing unit and/or in the connecting means for transporting aqueous liquid to the electrochemical chamber.

The term “a container” as used herein means a container or tank made of any suitable material, such as metal or plastics, that is adapted for containing saline water to be mixed with the municipal water.

The term “an isotonic water generator” as used herein is a commercially available device which generates isotonic water from municipal water.

The term “a control system” as used herein means a device or apparatus, or set of devices, that controls, manage, command, direct and/or regulates the behavior of one or more selected from the group consisting of the container, the mixing unit, the pumping means, the electrochemical chamber, the flow signal unit, the current supply, and the exit section.

The term “exit section” as used herein means the point where the aqueous liquid leaves the device of the present invention, when the device is in operation according to the present invention.

The term “an aqueous liquid” as used herein means a liquid comprising water and salts and is the result of the mixture of municipal water and saline water, which generates the aqueous liquid having a salt concentration which is lower than the salt concentration in the saline water.

The term “free available chlorine” as used herein and abbreviated FAC means all chlorine dissolved in the water, that is Cl₂, HOCl, and OCl⁻. FAC may be measured by portable water testing methods. Such water testing methods, such as DPD do not distinguish between these three species, and they constitute a pH dependant equilibrium. The total amount of FAC is an indicator of the disinfection capacity of the system. Typically, a test vial with color reading is used or a EPA approved strip.

The term “municipal water” as used herein means drinking water or tap water that live up to the latest WHO guidelines for drinking water quality

The term “saline water” as used herein means water having a salt concentration above 3% to saturated, wherein saline water having a salt concentration above 5% is termed brine. The salt or saline water referred to mainly contains NaCl, although other salts such as NaI in very low concentrations may be present.

The term “a mixing unit” as used herein means a device or set of devices that when in operation can mix municipal water and saline water to obtain the aqueous liquid containing a desired concentration of salt for entering the electrochemical chamber. A typical mixing unit is a T-element with a non-return valve.

The term “pumping means” as used herein means a pump or a means of transporting saline water from the container of the device to the mixing unit, such means may be by gravity, such as, for instance, if the saline water container is located above the mixing unit when the device is in operation and receives water from the municipal water supply.

The term “an electrochemical chamber comprising a pair of electrodes for providing electrolysis” as used herein means that the chamber has one or more pairs of electrodes, such as one or more pairs of electrode plates. Preferably the electrode is a pair of parallel and symmetrically arranged electrode plates, such as perforated electrode plates, made of a conductive material, such as expanded metal, and having a suitable distance for providing electrolysis. An example of such an electrode is disclosed in PCT/DK2009/000215. The conductive material is without limitation selected from a metal such as copper, aluminium, titanium, doped diamond, tin, silver, nickel, platinum, iron, lead, and oxides thereof, and alloys thereof. Typically, the electrode plates are made of titanium covered with ruthenium/iriduim oxid.

The term “a flow signal unit” as used herein means an instrument, such as a flow meter, used to measure linear, nonlinear, mass or volumetric flow rate of a liquid or a gas. Examples of flow meters suitable for use in the device of the present invention are selected from an impeller type, a mass flow meter, a magnetic type, a different gear type sensor, a coriolis type sensor.

The term “a sterile filter unit” as used herein means a filter that effectively removes microorganisms, such as a membrane filter with a with pore size 0.2 μm. Examples are Ceramic membranes, Hollow fiber PES filters, Teflon type membrane filters.

The term “a connecting means” as used herein means a liquid and air tight connection between two units, such as the mixing unit and electrochemical chamber, which is adapted for transportation of the aqueous liquid, such as tubes, pipes or the like made of plastic, metal or the like.

The term “a pre-filter unit” as used herein means a preliminary filter, such as the first filter in the device of the present invention which removes larger particles before the water is transported to the mixing unit.

The term “a water softener unit” as used herein means a unit that removes calcium, magnesium and other metal cations from the municipal water. Typically, the water softener unit is selected from an ion exchange unit, such as an ion exchange unit adapted to lower pH. Other water softeners are selected from the group consisting of a cartridge type generated with hydrochloric acid, the cartridge type generated with sodium chloride, a regenerative type with hydrochloric acid as regenerative agent, and a regenerative type with sodium chloride as regenerative agent. The term “an activated carbon filter unit” as used herein means a carbon filter that removes odors and colors from the water.

The term “a dosing section” as used herein means a section where specific and desired amounts and/or concentrations of aqueous liquid treated by electrolysis and sterile filtered are withdrawn from the device of the present invention. Examples are a water tap type, a spray gun type, a bottling device, an irrigation device.

The term “a user interface” as used herein means a computer, an ipad, a smart phone, a notebook, and a mac, incl monitor, as well as any other apparatus that makes it possible for a user to follow the process parameters of the device when in operation. The user interface is preferably an integrated part of the device of the present invention, but may also be remote.

The term “a backflow prevention device” as used herein means a device arranged before any pre-filter or the mixing unit of the device of the present invention which prevents any water from the pre-filter or mixing unit to flow back and get mixed with municipal water, such as a EN1717 conforming controllable non-return valve, a similar conforming vacuum breaker or an air gap. The term “a non-return valve means” as used herein means a check valve, a clack valve, a non-return valve or a one-way valve that allows liquid such as water and aqueous liquid to flow through it in only one direction. The non-return valve means has one opening for liquid to enter and one for liquid to leave.

The term “a flush system” as used herein means a device that when in operation cleanse the flow system before dosing.

The term “adjuvant wound therapy” as used herein means invasion/replication of microorganisms within a wound area, leading to cell injury and tissue damage. Examples of infections are mixed species biofilm, biofilm/infection with bacteria such as Pseudomonas aeruginosa, Staphylococcus aureus, Methicillin resistant Staphylococcus aureus, Beta-hemolytic streptococci, Coliform bacteria or Clostridium species

The present inventors have provided a device for killing bacteria described in PCT/DK2009/000215, containing a pair of parallel and symmetrically arranged perforated electrode plates, which electrodes are also suitable for use in the present invention. Thus, in a further embodiment the electrode comprises a pair of parallel and symmetrically arranged perforated electrode plates having a suitable distance, wherein each pair is optionally fitted with a fuse, wherein a suitable current density is applied, and wherein the plates are made of a conductive material and are arranged in a perpendicular plane. Typically, the pair of parallel and symmetrically arranged perforated electrode plates has/have a distance selected from 1-5 mm, such as 1, 2, 3, 4 or 5 mm and combinations thereof. The electrodes are arranged in pairs that may have the same distance between the plates or may have different distance between the plates, if more than one pair of electrodes is present. Typically, from 1 to 11 pairs of parallel and symmetrically arranged perforated electrode plates are present, such as 1-10, 2-9, 3-8, 4-7, or 5-6 pairs of parallel and symmetrically arranged perforated electrode plates.

When a pair of parallel and symmetrically arranged electrode plates, such as perforated plates, is used, such pair of parallel and symmetrically arranged electrode plates is optionally arranged such that in a perpendicular plane view 60-100% of the area of passage is inserted by the electrodes.

Typically, the current density is above 5 mA/cm², such as from 5 to 30 mA/cm². The device of the present invention can produce two types of aqueous liquid:

| | | | Theoretical | Preferable |
|---|---|---|---|---|
| | Water type | Purpose | range | range |
| Low | Normal saline with low | For use in situations where | 0.3-10 ppm | 0.5-3 ppm |
| FAC | FAC to keep the water | normal saline or tap water is | | |
| | clean from microorganisms | used - but is not for | | |
| | | intravenous use! | | |
| High | Normal saline with a | Reducing microorganisms | 50-200 ppm | 70-100 ppm |
| FAC | biocidal effect | on skin or in wounds by | | |
| | | cleansing, disinfection, | | |
| | | irrigation, moistening, | | |
| | | dressings, debridement | | |

In drinking water pH, can be within the range of 7-8.5
The preferable range is pH 5.5-7.0

| | | 70 ppm | 80 ppm | 100 pmm |
|---|---|---|---|---|
| | | FAC | FAC | FAC |
| | HOCl % at | HOCl | HOCl | HOCl |
| pH | 30° C. | (ppm) | (ppm) | (ppm) |
| 5 | 100 | 70* | 80* | 100* |
| 5.5 | 99 | 69* | 79* | 99* |
| 6 | 97 | 68* | 78* | 97* |
| 6.5 | 91 | 64* | 73* | 91* |
| 7 | 76 | 53* | 61* | 76* |
| 7.2 | 66 | 46* | 53* | 66* |
| 7.5 | 50 | 35# | 40* | 50* |
| 7.8 | 33 | 23# | 26# | 33# |
| 8 | 24 | 17** | 19** | 24# |
| 8.5 | 9 | 6** | 7** | 9** |
| 9 | 3 | 2** | 2** | 3** |

*Good biocidal effect and low cytotoxicity
#Effect will be lowered. It is sufficient in some situations and in others the effect could be too low, which is known to the skilled person
**The effect is not expected to be much different from normal saline. The risk of mild adverse events is increased.

### DRAWINGS

The invention will now be described more fully with reference to the appended drawings illustrating typical embodiments of the invention. These drawings are by no means limiting the scope of the present invention and are only intended to guide the skilled person for better understanding of the present invention.

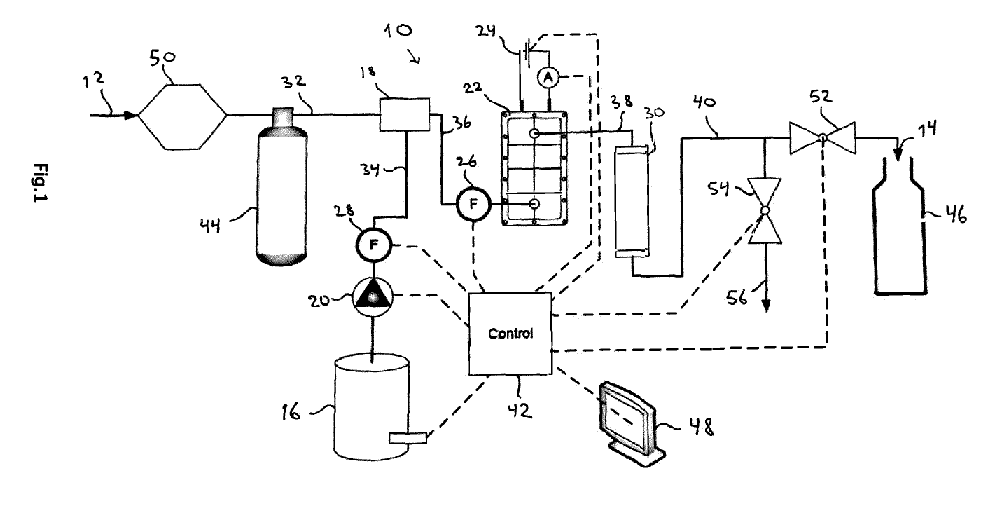

FIG. 1 illustrates a typical embodiment of the device of the present invention. When the device (10) is connected to water and current, municipal water is supplied via a connection (12) to a backflow prevention device (50), and further transported to a pre-filter (44). When leaving the pre-filter (44) the water is transported in a connection (32) to the mixing unit (18) where it is mixed with saline water from the container (16). Saline water is transported from the container (16) through a connection (34) by means of a pump (20). In the connection (34) is a flowmeter (28) which measures volume velocity of the saline water in the connection (34). In the mixing unit (18) the municipal water and saline water is mixed and then the aqueous liquid is transported via a connection (36) to an electrochemical chamber (22). Another flowmeter (26) measures volume velocity of the aqueous liquid in the connection (36). The electrochemical chamber (22) receives current from a current supply (24) and the aqueous liquid from the mixing unit (18) is subjected to electrolysis in the chamber (22). After electrolysis, the aqueous liquid now containing FAC is transported via a connection (38) to a sterile filter (30), and then transported via a connection (40) to an exit (14) which exit has a dosing section (46). A valve (52) is placed in the connection (40) to control the amount of liquid to the exit (14). A further valve (54) is placed so that aqueous liquid containing FAC can leave via a second exit (56). A control system (42) is in communication with the container (16), the mixing unit (18), the pump (20), the electrochemical chamber (22), the flow signal units (26, 28), the current supply (24), the exit section (14, 56) and a user interface (48) allowing a user to monitor the entire process when in operation.

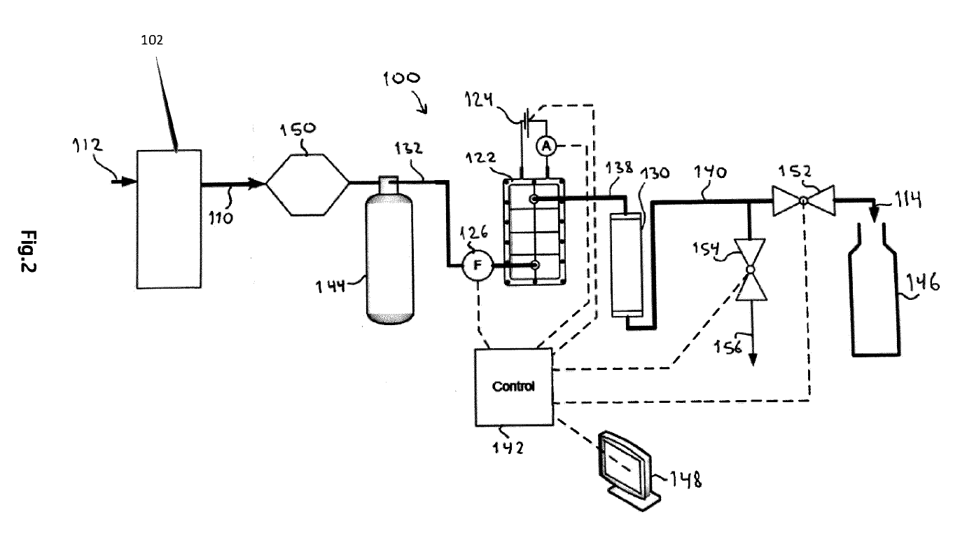

FIG. 2 illustrates another typical embodiment of the device of the present invention. When the device (100) is connected to water and current, municipal water is supplied via a connection (112) to an isotonic generator (102) and connection (110) then leads the isotonic water generated in (102) to the backflow prevention device (150), and further transported to a pre-filter (144). When leaving the pre-filter (144) the saline water is transported in a connection (132) to an electrochemical chamber (122). In the connection (132) is a flowmeter (126) which measures volume velocity of the saline water in the connection (132). The electrochemical chamber (122) receives current from a current supply (124) and the aqueous liquid from is subjected to electrolysis in the chamber (122). After electrolysis, the aqueous liquid now containing FAC is transported via a connection (138) to a sterile filter (130), and then transported via a connection (140) to an exit (114) which exit has a dosing section (146). A valve (152) is placed in the connection (140) to control the amount of liquid to the exit (114). A further valve (154) is placed so that aqueous liquid containing FAC can leave via a second exit (156). A control system (142) is in communication with the electrochemical chamber (122), the flow signal unit (126), the current supply (124), the exit section (114, 156) and a user interface (148) allowing a user to monitor the entire process when in operation.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

Recitation of ranges of values herein are merely intended to serve as a short method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about”, where appropriate).

All methods described herein can be performed in any suitable order unless otherwise indicated herein or otherwise clearly contradicted by context.

The terms “a” and “an” and “the” and similar referents as used in the context of de-scribing the invention are to be construed to insert both the singular and the plural, unless otherwise indicated herein or clearly contradicted by context. Thus, “a” and “an” and “the” may mean at least one, or one or more.

The term “and/or” as used herein is intended to means both alternatives as well as each of the alternatives individually. For instance, expression “the municipal water and/or saline water from the container” means “the municipal water and saline water from the container; the municipal water; or the saline water from the container”, all three alternatives are subject to individual embodiments.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter re-cited in the aspects or claims presented herein to the maximum extent permitted by applicable law.

The features disclosed in the foregoing description may, both separately and in any combination thereof, be material for realizing the invention in diverse forms thereof.

1-49. (canceled)

50. A device suitable for producing an aqueous liquid having a desired concentration of free available chlorine (FAC) wherein the device is adapted to receive municipal water from a municipal water supply, and for the aqueous liquid having the desired concentration of FAC to exit the device from an exit section, comprising:
a) a container adapted to contain saline water of a desired salt concentration,
b) a mixing unit adapted for mixing the saline water from the container with the municipal water,
c) a pumping means adapted to move the saline water from the container to the mixing unit,
d) or as an alternative to a)-c) an isotonic water generator,
e) an electrochemical chamber comprising a pair of electrodes for providing electrolysis of the aqueous liquid entering the chamber from the mixing unit, wherein the pair of electrodes are adapted to receive current from a current supply,
f) a flow signal unit for measuring a volume velocity of the aqueous liquid entering the electrochemical device and/or the mixing unit, and capable of providing volume velocity data, or if the velocity of the aqueous liquid and salt concentration hereof are fixed and known, then the flow signal unit may be omitted,
g) a sterile filter unit adapted to receive the aqueous liquid from the electrochemical chamber, and for the filtered aqueous liquid to exit the sterile filter unit,
h) (i) a connecting means for transporting municipal water from the supply to the mixing unit, for transporting saline water to the mixing unit, for transporting mixed water to the electrochemical chamber, for transporting the aqueous liquid from the electrochemical chamber to the sterile filter unit, and for transporting the aqueous liquid from the sterile filter to the exit section, or alternatively (ii) in d) a connecting means for transporting isotonic water to the electrochemical chamber, for transporting the aqueous liquid from the electrochemical chamber to the sterile filter unit, and for transporting the aqueous liquid from the sterile filter to the exit section and
j) a control system adapted to communicate with one or more selected from the group consisting of the container, the mixing unit, the pumping means, the electrochemical chamber, the flow signal unit, the current supply, and the exit section.

51. The device of claim 50 suitable for producing an aqueous liquid having a desired concentration of free available chlorine (FAC) wherein the device is adapted to receive municipal water from a municipal water supply, and for the aqueous liquid having the desired concentration of FAC to exit the device from an exit section, comprising:
i) an isotonic water generator,
ii) an electrochemical chamber comprising a pair of electrodes for providing electrolysis of the aqueous liquid entering the chamber from the mixing unit, wherein the pair of electrodes are adapted to receive current from a current supply,
iii) a flow signal unit for measuring a volume velocity of the aqueous liquid entering the electrochemical device and/or the mixing unit, and capable of providing volume velocity data, or if the velocity of the aqueous liquid and salt concentration hereof are fixed and known, then the flow signal unit may be omitted,
iv) a sterile filter unit adapted to receive the aqueous liquid from the electrochemical chamber, and for the filtered aqueous liquid to exit the sterile filter unit,
v) a connecting means for transporting isotonic water to the electrochemical chamber, for transporting the aqueous liquid from the electrochemical chamber to the sterile filter unit, and for transporting the aqueous liquid from the sterile filter to the exit section, and
vi) a control system adapted to communicate with one or more selected from the group consisting of the electrochemical chamber, the flow signal unit, the current supply, and the exit section.

52. The device of claim 50 suitable for producing an aqueous liquid having a desired concentration of free available chlorine (FAC) wherein the device is adapted to receive municipal water from a municipal water supply, and for the aqueous liquid having the desired concentration of FAC to exit the device from an exit section, comprising:
a) a container adapted to contain saline water of a desired salt concentration,
b) a mixing unit adapted for mixing the saline water from the container with the municipal water,
c) a pumping means adapted to move the saline water from the container to the mixing unit,
d) an electrochemical chamber comprising a pair of electrodes for providing electrolysis of the aqueous liquid entering the chamber from the mixing unit, wherein the pair of electrodes are adapted to receive current from a current supply,
e) a flow signal unit for measuring a volume velocity of the aqueous liquid entering the electrochemical device and/or the mixing unit, and capable of providing volume velocity data,
f) a sterile filter unit adapted to receive the aqueous liquid from the electrochemical chamber, and for the filtered aqueous liquid to exit the sterile filter unit,
g) a connecting means for transporting municipal water from the supply to the mixing unit, for transporting saline water to the mixing unit, for transporting mixed water to the electrochemical chamber, for transporting the aqueous liquid from the electrochemical chamber to the sterile filter unit, and for transporting the aqueous liquid from the sterile filter to the exit section, and
h) a control system adapted to communicate with one or more selected from the group consisting of the container, the mixing unit, the pumping means, the electrochemical chamber, the flow signal unit, the current supply, and the exit section.

53. The device of claim 50, further comprising:
a pre-filter unit adapted to receive the municipal water, and connecting means for transporting the filtered municipal water to the mixing unit.

54. The device of claim 50, further comprising:
a water softener unit, wherein the water softener unit is adapted to receive filtered municipal water, and connecting means for transporting the softened and filtered municipal water to the mixing unit.

55. The device of claim 54, wherein the water softener unit is selected from an ion exchange unit, such as an ion exchange unit adapted to lower pH.

56. The device of claim 50, further comprising:
a dosing section at the exit section or a dosing section remote from the exit section and connecting means for transporting the aqueous liquid from the exit section to the dosing section.

57. The device of claim 50 adapted to operate at a volume velocity from 0.5 to 10 L aqueous liquid per minute, such as from 0.5 to 2 L aqueous liquid per minute.

58. The device of claim 50, wherein a flush system is incorporated in the dosing section.

59. The device of claim 50, wherein the flow signal unit is located between the mixing unit and the electrochemical device.

60. The device of claim 50, wherein the electrochemical chamber is without any membrane.

61. A method of preparing an aqueous liquid having a desired concentration of free available chlorine (FAC) comprising the steps of,
a) supplying municipal water to the device of claim 50, wherein the device contains saline water in the container, or the isotonic water generator is present,
b) supplying current to the device,
c) mixing municipal water and saline water in the mixing unit, and leading the aqueous liquid to the electrochemical chamber, or supplying municipal water to the isotonic water generator,
d) adjusting water and liquid flows, and dosing to provide the aqueous liquid having the desired concentration of FAC, and
e) collecting the aqueous liquid having the desired concentration of FAC at the exit section.

62. The method of claim 61, wherein the concentration of FAC at the exit section is selected from 0.3 ppm to 1000 ppm, such as from 0.3 ppm to 200 ppm, such as from 10 ppm to 120 ppm.

63. The method of claim 61, wherein pH of the water and the aqueous liquid in the device is between 5 and 8, such as between 5.5 and 7.5.

64. The method of claim 63, wherein pH of the water and the aqueous liquid in the device is between 5.5 and 7.

65. The method of claims 61, wherein the volume velocity of the device is adjusted to from 0.5 to 10 L aqueous liquid per minute, such as from 0.5 to 2 L aqueous liquid per minute, such as 1 L/minute.

66. The method of claim 61, wherein the salt concentration of the saline water is from 10% w/w to saturated, such as 15% w/w to saturated.

67. The method of claim 61, wherein the current is delivered in pulses, where the peak current is 10 A and the mean current, for the volume velocity of 0.5 to 2 L aqueous liquid per minute, is 5-7 A and the voltage is 6-10V, such as 8V, to deliver the aqueous liquid having the desired concentration of 100 ppm FAC.

68. The method of claim 61, wherein the municipal water from the supply is drinking water according to latest WHO guidelines for drinking water quality.

69. The method of claim 61, wherein the municipal water and/or saline water from the container and/or aqueous liquid is/are heated above room temperature during the preparation, such as by mixing water and saline water by different temperatures or by using a heating system for heating the aqueous liquid before entering the electrochemical chamber.

70. The method of claim 61, wherein the temperature of the aqueous liquid leaving the electrochemical chamber is in the range of 30° C.-35° C.

71. The method of claim 61, wherein the water softener unit additionally is used to lower the pH of the municipal water.

72. The method of claim 61, wherein the additional flow signal unit located between the container and the mixing unit is used to partly control the dosing speed of the saline water from the container and the electrical current used for the electrolysis, and wherein automated setting or user input is used to partly control the dosing speed of the saline water from the container and the electrical current used for the electrolysis.

73. The method of claim 61, wherein voltage and/or current is measured in the electrochemical chamber, using the constant characteristics of the chamber to calculate the conductivity of the aqueous liquid passing through the chamber.

74. The method of claim 73, wherein a constant conductivity of the aqueous liquid is maintained by partly controlling the dosing speed of the saline water from the container and the electrical current used for the electrolysis, and wherein automated setting or user input is used to partly control the dosing speed of the saline water from the container and the electrical current used for the electrolysis.

75. The method of claim 61, wherein a flush system is incorporated in the dosing section, the flush system automatically leading a predetermined amount of aqueous liquid, such as 10-250 mL of aqueous liquid, to drain before allowing a user, such as a physician or nurse, to draw the aqueous liquid.

76. The method of claim 61, wherein the container and pumping means in operation produce a selected range of salt concentrations in the aqueous liquid leaving the mixing unit from 0.01% w/w to 0.95% w/w at a water flow of 1 L/minute.

77. The method of claim 61, wherein the current applied to the electrolysis and timing generates from 40-200 ppm of chlorine, such as 40-120 ppm, in a normal saline liquid (0.9% NaCl), wherein the concentration (ppm) of chlorine is selected and kept constant ±10%.

78. The method of claim 61, wherein the current applied to the electrolysis and timing generates from 1-40 ppm of chlorine in an aqueous liquid containing 0.01-0.25% salt (NaCl), wherein the concentration (ppm) of chlorine is selected and kept constant ±75%.

79. The method of claim 61, wherein the municipal water supplied is drinking water which is mixed with saline water from the container, wherein the saline water comprises high-purity sodium chloride dissolved in mineral-free water.

80. The method of claim 61, wherein the municipal water supplied is drinking water which is mixed with saline water from the container, wherein the saline water comprises various salts and the device is adjusted to an osmotic pressure of approximately 308 mosmol/L, using a conversion from conductivity to osmotic pressure, where the error from non-sodium and non-chloride salts present constitute less than 1%.

81. The method of claim 61, wherein the current is regulated to a constant level using a constant current generator circuit, and the voltage varies with the area of the pair of electrodes, such as from 3.7-5.7V, and the current is delivered in pulses.
